(19) United States
(12) Patent Application Publication
(10) Pub. No.: US 2026/0077099 A1
(43) Pub. Date: Mar. 19, 2026

(54) Methods of Treating an Eye Disorder

(71) Applicants: Universiteit Antwerpen, Antwerpen (BE); Universitair Ziekenhuis Antwerpen, Edegem (BE)

(72) Inventors: Bert van den Bogerd, Wilrijk (BE); Sorcha Ní Dhubhghaill, Edegem (BE)

(73) Assignees: Universiteit Antwerpen, Antwerpen (BE); Universitair Ziekenhuis Antwerpen, Edegem (BE)

(21) Appl. No.: 18/870,744

(22) PCT Filed: Apr. 26, 2023
(86) PCT No.: PCT/EP2023/060961

§ 371 Date: Dec. 2, 2024

Related U.S. Application Data
(60) Provisional application No. 63/348,099, filed on Jun. 2, 2022.

(51) Int. Cl. A61L 27/22; A61L 27/52; A61L 27/58

(52) U.S. Cl. CPC A61L 27/222; A61L 27/52; A61L 27/58; A61L 2400/06; A61L 2430/16

(57) ABSTRACT

The invention provides methods of treating an eye disorder and compositions for use therein, the methods comprising applying a crosslinkable liquid composition onto an anterior corneal surface of said eye using a mold; crosslinking the crosslinkable liquid composition on the anterior corneal surface of the eye, thereby obtaining a crosslinked composition on the anterior corneal surface of the eye; and correcting the curvature of the crosslinked composition.

### FIELD

The invention is broadly applicable in the medical field and more specifically concerns methods of treating an eye disorder, like a refractive error or a chronic or subacute corneal disease involving an irregularity of the cornea.

### BACKGROUND

The cornea is the transparent tissue in the front of the eye. It functions as the window of the eye and is responsible for two thirds of the refraction of the incoming light, while the remaining refraction is done by the crystalline lens. Proper refraction of incoming light is necessary to project a clear image onto the retina, where light is converted to electrical signals that are transmitted via the optic nerve to the visual cortex in the brain. Refractive disorders, such as myopia, hyperopia, astigmatism and presbyopia, can blur vision for various distances and are, in most cases, the result of an aberrant corneal curvature. A lens can be placed in front of the eye as a correction, and that can either be in the shape of contact lenses or spectacles, to compensate the aberrant light path. Uncorrected refractive errors can lead to (severe) visual impairment and, secondarily, to headaches, fatigue and eye irritation. That is why refractive errors are listed as the most prevalent cause of reversible blindness worldwide.

Another solution to correcting refractive disorders is to reshape the cornea by means of refractive laser surgery, termed laser ablation or photoablation, of which different techniques exist including photorefractive keratectomy (PRK), laser assisted in situ keratomileusis (LASIK) and small incision lenticule extraction (SMILE). The common denominator of these procedures is that they aim to adjust the curvature of the cornea by means of ablation, i.e. removing corneal tissue by means of a laser pulse. This tissue dissection will result in change of the corneal curvature and thus correcting the corneal focusing power to properly focus light onto the retina. In the case of hyperopia, the central optical zone is relatively steepened by applying laser pulses to the corneal midperiphery, while in myopia the central cornea is flattened to reduce the curvature.

Photoablative treatment for refractive errors, like PRK, LASIK and SMILE, have limitations as they are all fundamentally subtractive. Since tissue is removed from the cornea to relatively steepen or flatten the curvature, the most obvious limitation is the extent of the tissue removal required to achieve the effect. Removing too much corneal tissue renders a thin cornea that is prone to ectasia (corneal thinning) and corneal perforation. For hyperopia, photo-ablative laser surgery is safe and effective up to +2 dioptres (dpt) in practice, but becomes less predictable in higher degrees. This is also the case for astigmatism up to 3 dpt. Secondly, the effect of refractive surgery (for hyperopia or presbyopia) tends to regress over time since epithelial cells overgrow the induced grooves in the cornea, thereby returning to a state similar to its initial aberrant curvature. Thirdly, refractive surgery has, depending on the specific technique, risks associated as a result of suboptimal procedure such as risk of corneal haze and long visual recovery periods (PRK), flap related problems (LASIK) or complications due to challenging technical methods (SMILE).

There remains a need for new methods to treat these eye disorders, like refractive errors.

### SUMMARY

The present invention is at least in part based on the inventors'innovative insight and experimental evaluation that a refractive error or a chronic or subacute corneal disease or disorder involving an irregularity of the cornea can be treated by applying a crosslinkable composition onto the anterior corneal surface of the affected eye as a liquid using a mold, subsequently crosslinking the composition in situ. It is then possible, when necessary to correct the curvature of the newly formed corneal onlay, using a technique such as photoablation, without subtracting the stroma volume from the cornea per se. The advantage of crosslinking the tissue in situ is the simultaneous polymerization of the hydrogel and adherence to the cornea through chemical interaction. Additionally, as the uncrosslinked biomaterial is liquid, it perfectly fits the patient's corneal geometry compared to prefabricated onlays.

The methods and crosslinkable compositions for use in the methods of the present invention provide a long-term, but reversible, solution for treating eye disorders, such as a refractive error or a chronic or subacute corneal disease involving an irregularity of the cornea. There is very limited risk of post-procedure complications such as dry eye disease or post operative pain, as the corneal stromal tissue is not affected, neither are the corneal nerves are damaged. Moreover, the methods and crosslinkable compositions for use in the methods of present invention provide a wider therapeutic window than subtractive refractive laser surgery as the biomaterial is added to the corneal surface, and the method is not dependent on the thickness of the cornea itself. For example, higher corrections are possible for hyperopia and presbyopia, irregular corneas can be treated, and higher astigmatism corrections than that with just laser can be achieved. Additionally, the method allows for restoring/recovering a partly or completely damaged cornea resulting from chronic or acute corneal diseases.

Accordingly, the invention provides a crosslinkable liquid composition for use in the treatment of an eye disorder in an eye of a subject, wherein the treatment comprises a method comprising the steps of applying said crosslinkable liquid composition onto an anterior corneal surface of said eye comprising said eye disorder; crosslinking the crosslinkable liquid composition on the anterior corneal surface of the eye, thereby obtaining a crosslinked composition on the anterior corneal surface of the eye; and optionally, correcting the curvature of the crosslinked composition; wherein the eye disorder is a refractive error or a chronic or subacute corneal disease involving an irregularity of the cornea, and wherein the method comprises introducing said crosslinkable liquid composition into a mold which is positioned onto the anterior corneal surface of the eye.

Similarly, the invention provides a method of treating an eye disorder in an eye of a subject, comprising the steps of applying a crosslinkable liquid composition onto an anterior corneal surface of said eye comprising said eye disorder; crosslinking the crosslinkable liquid composition on the anterior corneal surface of the eye, thereby obtaining a crosslinked composition on the anterior corneal surface of the eye; and optionally correcting the curvature of the crosslinked composition; wherein the eye disorder is a refractive error or a chronic or subacute corneal disease involving an irregularity of the cornea, and wherein the crosslinkable composition is applied onto the anterior corneal surface of the eye using a mold. More particularly, the method comprising either introducing said crosslinkable liquid composition into said mold before the mold is placed onto said anterior corneal surface of the eye or placing said mold onto said anterior corneal surface of the eye and introducing said crosslinkable liquid composition into said mold.

In particular embodiments of the crosslinkable liquid composition for use or the methods of the invention, the mold is a corneal vacuum suction device, a corneal bath or a contact lens.

In particular embodiments the anterior corneal surface is debrided at least in part of corneal epithelial cells before or after positioning of the mold on the corneal surface.

In particular embodiments of the crosslinkable liquid composition for use or the methods of the invention, the method comprises removing the mold after crosslinking the crosslinkable composition.

In particular embodiments of the crosslinkable liquid composition for use or the methods of the invention, the crosslinkable liquid composition is applied as a single layer. In particular embodiments, more particularly where the crosslinkable liquid composition is applied onto a lens which is then placed on the surface of the eye, this implies that the crosslinkable liquid composition is applied as a single layer into the mold. In particular embodiments, more particularly where the crosslinkable liquid composition is applied in an o-ring which is placed on the eye, this implies that the crosslinkable liquid composition is applied as a single layer onto the anterior corneal surface of the eye.

In particular embodiments of the crosslinkable liquid composition for use or the methods of the invention, the crosslinkable liquid composition comprises a crosslinkable biomaterial and optionally one or more photoinitiators. More particularly, the crosslinkable biomaterial is selected from the group consisting of protein-based polymers, polysaccharide-based polymers and synthetic polymers.

In particular embodiments of the crosslinkable liquid composition for use or the methods of the invention, the crosslinking is performed by photocrosslinking, by exposure to O₂ or by one or more enzymes, preferably by photocrosslinking such as by UV irradiation.

In particular embodiments of the crosslinkable liquid composition for use or the methods of the invention, after cross-linking the crosslinked composition has a diameter of from 6.0 to 9.0 mm and a thickness of from 20.0 μm to 400.0 μm prior to correcting the curvature of the crosslinked composition.

In particular embodiments of the crosslinkable liquid composition for use or the methods of the invention, the crosslinked composition is resistant to biodegradation for a period of at least 6 months, preferably at least 12 months.

In particular embodiments, the refractive error is selected from the group consisting of myopia, hyperopia, astigmatism and presbyopia.

In particular embodiments, the chronic or subacute corneal disease involving an irregularity of the cornea is selected from the group consisting of corneal ulcer, corneal erosion, a corneal ectatic disorder or a corneal irregularity caused by trauma or epithelial basement membrane dystrophy.

These and further aspects and preferred embodiments of the invention are described in the following sections and in the appended claims. The subject-matter of the appended claims is hereby specifically incorporated in this specification.

### BRIEF DESCRIPTION OF DRAWINGS

FIG. 1. The crosslinked material is difficult to remove from a corneal surface using forceps.

FIG. 2 Optical coherence tomography (OCT) imaging reveals the adherence of methacrylated gelatin onto the cornea with a mean thickness of around 1000 μm.

FIG. 3 shows that the gel fraction of GelMA increased with polymer concentration due to the increased polymer density for crosslinking.

FIG. 4 shows that the water uptake for GelMA in different concentrations with different photoinitiators. Hydrogels with the lowest concentration of GelMA exhibited the highest water uptake and vice versa, which can be explained by the increased gel fraction and thus viscosity of the hydrogel with increasing polymer concentration. All concentrations reached an equilibrium after 4 hours.

FIG. 5 shows that the transparency of GelMA is at least 75% when measured over the visual spectrum. No significant transparency difference was observed for any polymer or photoinitiator concentration.

FIG. 6 The different steps of an exemplary method of the invention for the treatment of a hypermetropic cornea (farsightedness).

FIG. 7 The different steps of an exemplary method of the invention for the treatment of a hypermetropic cornea (farsightedness) using an o-ring.

FIG. 8 The refractive correction by a corneal onlay versus conventional laser refractive surgery.

FIG. 9 Photograph of a corneal onlay provided with an o-ring (left) and a lens (right).

FIG. 10 Comparison of refractive correction according to traditional methods with that according to the methods of the invention.

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . .” or the expression “between . . . and . . . ”or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately”refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art. For example, in the appended claims, any of the claimed embodiments can be used in any combination.

The present invention is at least in part based on the inventors'innovative insight and experimental evaluation that a refractive error or a chronic or subacute corneal disease involving an irregularity of the cornea can be treated by applying a crosslinkable composition onto the anterior corneal surface of the affected eye as a liquid using a mold for holding the crosslinkable composition while applying it to the anterior corneal surface, subsequently crosslinking the composition in situ and, optionally, correcting the curvature of the newly formed corneal onlay, such as by photoablation, without subtracting the stroma of the cornea per se. Accordingly, the method of present invention is less invasive as subtractive refractive laser surgery, such as PRK, LASIK and SMILE.

The methods and crosslinkable liquid composition for use in the methods of the present invention provide an integrated, long-term, but reversible, solution for treating eye disorders, such as a refractive error or a chronic or subacute corneal disease involving an irregularity of the cornea, and has several advantageous over known methods for treating such eye disorders.

For example, unlike glasses or contact lenses, the compositions for use and methods of present invention do not obstruct the subject during heavy duty work or contact sport, the crosslinked composition on the cornea obtained by the method present invention does not feel uncomfortable for the subject and, once applied, provides a long-term treatment of the eye disorder without risk of severe bacterial eye infections due to poor lens hygiene.

Furthermore, compared to subtractive refractive laser surgery, such as PRK, LASIK and SMILE, the method of the present invention has a very limited risk to post-procedure complications such as dry eye disease, post-operative pain or flap related problems, as the corneal stromal tissue is not affected nor is an epithelial flap is created. Moreover, the method of present invention provides a wider therapeutic window than subtractive refractive laser surgery as biomaterial is added to the corneal surface and the method is not dependent on the thickness of the cornea itself. For example, higher corrections are possible for, for example, hyperopia and presbyopia, irregular corneas can be treated and high astigmatism correction can be achieved.

The methods and compositions of the present invention particularly aims at treating a subject's eye disorder which was already present prior to treating the subject with the method as taught herein and not to treat a refractive error caused by the presence of the crosslinked material applied by the method as taught herein.

Accordingly, the invention provides crosslinkable liquid compositions for use in the treatment of a disorder of an eye, wherein the crosslinkable compositions are used in a particular way, i.e. they are applied using a mold. More particularly, the invention provides crosslinkable liquid compositions for use in a method of treatment of an eye disorder, wherein the method comprises the steps of applying said crosslinkable liquid composition onto an anterior corneal surface of said eye into a mold positioned on the eye; crosslinking the crosslinkable liquid composition on the anterior corneal surface of the eye, thereby obtaining a crosslinked composition on the anterior corneal surface of the eye. Optionally, the method additionally compr; and optionally, correcting the curvature of the crosslinked composition.

Similarly, the invention provides a method of treating an eye disorder in an eye of a subject in need of treatment, comprising applying a crosslinkable liquid composition onto an anterior corneal surface of said eye comprising said eye disorder using a mold; crosslinking the crosslinkable liquid composition on the anterior corneal surface of the eye, thereby obtaining a crosslinked composition on the anterior corneal surface of the eye; and optionally correcting the curvature of the crosslinked composition.

In particular embodiments, the compositions and methods of the invention are envisaged for use the treatment of an eye disorder which is a refractive error or a chronic or subacute corneal disease involving an irregularity of the cornea, preferably a refractive error of the cornea.

Reference to “therapy” or “treatment” encompasses curative treatments, and the terms may particularly refer to the alleviation or measurable lessening of one or more symptoms or measurable markers of a pathological condition such as a disease or disorder or a dysfunction (e.g. as a result of trauma or surgery). Measurable lessening includes any statistically significant decline in a measurable marker or symptom. Generally, the terms encompass both curative treatments and treatments directed to reduce symptoms and/or slow progression of the disease.

The term eye disorder thus generally encompasses both conditions caused by disease and conditions caused by other factors such as trauma or operation, which can affect the normal functioning of the eye.

The terms “subject”, “individual” or “patient” are used interchangeably throughout this specification, and typically and preferably denote humans, but may also encompass reference to non-human animals, preferably warm-blooded animals, even more preferably non-human mammals. Particularly preferred are human subjects including both genders and all age categories thereof. In other embodiments, the subject is an experimental animal or animal substitute as a disease model. The term does not denote a particular age or sex.

The term “subject in need of treatment” or similar as used herein refers to subjects diagnosed with or having a disease or disorder as recited herein.

The term “refractive error” or “refraction error” as used herein refers to an eye disorder wherein the shape of the eye and/or cornea prevents light from focusing correctly on the retinal fovea leading to impaired vision. Non-limiting examples of refractive errors are nearsightedness (myopia), farsightedness (hyperopia), regular and irregular astigmatism, and presbyopia. Symptoms caused by refractive errors include, but are not limited to, double vision, hazy vision, seeing a glare or halo around bright lights, squinting, headaches and eye strains. A refractive error may be diagnosed by any means known in the art, such as by use of an automated refractor. In preferred embodiments, the refractive error is of primary cause (e.g. naturally occurring or non-surgically induced) or of secondary cause which may include, but not limited to surgically induced (e.g. post-LASIK ectasia) as a result from infection, genetic factors, degenerative eye diseases or trauma to the eye.

In particular embodiments, the refractive error is selected from the group consisting of myopia, hyperopia, astigmatism (e.g. regular or irregular astigmatism) and presbyopia.

The term “myopia” or “nearsightedness” as used herein has its meaning as generally accepted in the art. In myopia or nearsightedness, light rays are brought to focus in front of the retina. This may occur because the focusing power of the cornea and lens is very high and/or because the eyeball is too long from front to back. For myopia, subtractive photo-ablative laser surgery is safe and effective up to-10 diopters (dpt) in practice, but becomes less predictable in higher degrees of myopia and display higher complication rates. Accordingly, in particular embodiments, the refractive error is myopia with a diopter of more than −10.0, such as from −10.5 to −20.0.

The term “hyperopia” or “farsightedness” as used herein has its meaning as generally accepted in the art. In hyperopia or farsightedness, light rays are brought to focus behind the retina. This may occur because the focusing power of the cornea and lens is very low and/or because the eyeball is short in length from front to back. For hyperopia, subtractive photo-ablative laser surgery is safe and effective up to 3 diopters (dpt) in practice, but becomes less predictable in higher diopters. Accordingly, in particular embodiments, the refractive error is hyperopia with a diopter of more than +3.0, such as from +3.5 to +10.0, from +3.5 to +8.0, preferably from +3.5 to +4.0, +3.5, or +4.0.

The term “presbyopia” or “age-related farsightedness” as used herein has its meaning as generally accepted in the art. Presbyopia is physiological insufficiency of accommodation associated with the aging of the eye that results in progressively worsening ability to focus clearly on close objects. Presbyopia typically occurs due to age related changes in lens (decreased elasticity and increased hardness) and ciliary muscle of the eye, causing the eye to focus light behind rather than on the retina, when looking at close objects.

The term “astigmatism” as used herein has its meaning as generally accepted in the art, and includes regular, irregular astigmatism, simple astigmatism, compound astigmatism, myopic astigmatism, hyperopic astigmatism, mixed astigmatism, lenticular astigmatism, and corneal astigmatism, xxx. Astigmatism is a refractive error in the eye due to rotational asymmetry in the eye's refractive power. The underlying mechanism involves an irregular curvature of the cornea or abnormalities in the lens of the eye. In eyes without astigmatism, the cornea and lens have a more or less similar curvature in all directions. This allows light to be focused to a single point on the retina. People with astigmatism have more curvature in one direction, or meridian, than in another, so that light is not able to focus on a single point on the retina. This results in blurring of vision at all distances. For astigmatism, subtractive photo-ablative laser surgery is safe and effective up to 3 diopters (dpt) in practice, but becomes less predictable in higher diopters. Accordingly, in particular embodiments, the refractive error is astigmatism with a diopter of more than +3.0, such as from +3.5 to +16.0, from +3.5 to +8.0, preferably from +3.5 to +5.0, like +3.5, +4.0, +4.5, or +5.0. The corneal astigmatism may be corneal surgery-induced astigmatism or corneal transplantation-induced astigmatism.

In particular embodiments, the eye disorder is a chronic or subacute corneal disease involving an irregularity of the cornea. Non-limiting examples of chronic or subacute corneal disease involving an irregularity of the cornea comprise corneal ulcers (e.g. caused by trauma or inflammation), corneal erosion, corneal ectactic disorders (i.e. corneal thinning) such as keratoconus, keratoglobus or post-LASIK ectasia, or a corneal irregularity inducing an irregular astigmatism such as a result from trauma or epithelial basement membrane dystrophy.

In particular embodiments, the chronic or subacute corneal disease involving an irregularity of the cornea is selected from the group consisting of corneal ulcer, corneal erosion, a corneal ectatic disorder or a corneal irregularity caused by trauma or epithelial basement membrane dystrophy. Preferably, the corneal ectasia is keratoconus.

The terms “corneal ectasia” or “corneal ectatic disorder” as used herein refers to a group of uncommon, noninflammatory eye disorders characterised by bilateral thinning of the central, paracentral, or peripheral cornea.

In embodiments, the corneal ectasia may be selected form the group consisting of keratoconus, keratoglobus, pellucid marginal degeneration, posterior keratoconus, post-LASIK ectasia, and Terrien's marginal degeneration.

The term “keratoconus” refers to a progressive, noninflammatory, bilateral, asymmetric disease, characterized by paraxial stromal thinning and weakening that leads to corneal surface distortion.

In embodiments of the uses or methods as taught herein, the chronic or subacute corneal disease involving an irregularity of the cornea may be caused by an invasive treatment for correcting an eye disorder. Hence, in embodiments, the chronic or subacute corneal disease involving an irregularity of the cornea may be caused by refractive laser surgery, such as PRK, LASIK or SMILE.

In alternative embodiments, the chronic or subacute corneal diseases are diseases that were not caused by an invasive treatment for correcting an eye disorder. Accordingly, in particular embodiments, the chronic or subacute corneal disease involving an irregularity of the cornea is not caused by refractive laser surgery, such as PRK, LASIK or SMILE.

Likewise, in particular embodiments, the subject did not receive any invasive treatment for correcting the eye disorder in the eye, such as a refractive error, prior to the application of the crosslinkable liquid composition. In more particular embodiments, the subject did not receive refractive laser surgery, such as PRK, LASIK or SMILE, prior to the application of the crosslinkable liquid composition onto the anterior corneal surface of the eye.

Of particular interest are those subjects of which the eye disorder cannot be treated using existing techniques, such as subjects that are excluded from treatment by refractive laser therapy, such as subjects with a very thin cornea, subjects with persistent dry eyes or subjects performing contact sports. Accordingly, in particular embodiments, the subject has a cornea thickness of less than 480.0 μm, less than 450.0 μm or less than 400.0 μm.

Crosslinking is the formation of chemical links between molecular chains to form a three-dimensional network of connected molecules. Crosslinks may be formed by chemical reactions that occur spontaneously or are initiated by, for example, one or more enzymes, heat, pressure, change in pH, or irradiation. These chemical reactions may also be initiated by the presence of one or more crosslinking agents, which typically comprises multiple functional groups.

The crosslinkable liquid composition to be used in the method of present invention may comprise any type of biocompatible material (e.g. biomaterial) that is able to crosslink (spontaneously or upon induction, such as by UV irradiation) and can take up substantial amounts of water from its surroundings without dissolving at body temperature, such at about 37° C.

The “crosslinkable liquid” or “crosslinkable liquid composition” as referred to herein comprises a crosslinkable biomaterial. The crosslinkable biomaterial may be synthetic, non-synthetic or a combination thereof. The terms “liquid” or “liquid composition” in the context of the present invention encompass both completely liquid and semi-liquid compositions, i.e. include compositions which have a consistency between solid and liquid.

In particular embodiments, the crosslinkable biomaterial is selected from the group consisting of protein-based polymers, polysaccharide-based polymers, synthetic polymers, or a combination thereof.

Protein-based polymer may be collagen such as recombinant mammal collagen or mammal-sourced collagen, gelatin, fibrinogen, silk fibroin, or mussel inspired 3,4-dihydroxyphenyl-L-alanine (DOPA) polymers, but should not be considered to be limited thereto. In particular embodiments, the collagen is selected from the group consisting of collagen type I, type III, type IV, type V, type VII, and combinations thereof.

Non-limiting examples of polysaccharide-based polymers include hyaluronic acid or alginate. Gelatin-based biomaterials may be GelCORE such as described in Ehsan Shirzaei Sani et al., Sutureless repair of corneal injuries using naturally derived bioadhesive hydrogels, Science Advances, 2019, Vol. 5, no. 3, or methacrylated thermoresponsive COMatrix such as described in Ghasem Yazdanpanah et al., A light-curable and tunable extracellular matrix hydrogel for in situ suture-free corneal repair, Advanced Functional Materials, 2022.

Non-limiting examples of synthetic polymers include polyethylene glycol (PEG), poly(e-caprolactone) (PCL) and poly(vinylalcohol). Synthetic polymers may be polyethylene glycol-collagen such as described in Yoon Hong Chun et al., In vivo biocompatibility evaluation of in situ-forming polyethylene glycol-collagen hydrogels in corneal defects, Scientific Reports, 2021.

In particular embodiments, the crosslinkable biomaterial is functionalized using methacrylate, diacrylate, diacrylamide, xxxx. Accordingly, in particular embodiments, the crosslinkable biomaterial comprises, consists essentially of, or consists of one or more of 2-(hydroxyethyl)methacrylate (HEMA), 2-methacryloyl-oxyethyl phosphorylcholine (MPC), HEA, methyl methacrylate (MMA), methacrylic acid (MAA), polyethylene glycol dimethacrylate, polyethylene glycol diacrylate, and polyethylene diacrylamide.

In particular embodiments, the crosslinkable biomaterial does not have a transparency of less than 50%, when measured over the visual spectrum (400-750 nm), when crosslinked. In particular embodiments, the crosslinkable biomaterial is not dextran methacrylate.

The concentration of the crosslinkable biomaterial in the crosslinkable liquid composition may vary, as well as its degree of substitution.

In particular embodiments, the crosslinkable liquid composition comprises at least 1.0% (w/v), at least 5.0% (w/v), at least 10.0% (w/v), at least 15.0% (w/v), or at least 20.0% (w/v) of crosslinkable biomaterial. In particular embodiments, the crosslinkable liquid composition comprises from 1.0% (w/v) to 30.0% (w/v), such as from 1.0% (w/v) to 20.0% (w/v), of crosslinkable biomaterial. In particular embodiments, the crosslinkable liquid composition comprises 20.0% (w/v) of crosslinkable biomaterial.

The crosslinkable liquid composition may further also comprise one or more crosslinking agents and/or one or more photoinitiators, that participate in the crosslinking reaction.

Photoinitiators are compounds that upon radiation of light decompose into reactive species that activate polymerization of specific functional groups on the crosslinkable biomaterial. Accordingly, photoinitiators are typically used herein when the crosslinkable liquid composition is capable of being crosslinked by photocrosslinking. The type of photoinitiator as well as the concentration thereof can be varied in the crosslinkable liquid composition as intended herein. Each specific photoinitiator is typically linked to an excitation wavelength spectrum, of which the peak of the spectrum is the most optimal wavelength to create radicals upon excitation. Non-limiting examples of photoinitiators that may be used in the method as taught herein include riboflavin, indocyanine green, Janus green, rose Bengal, methylene blue, sodium persulphate, ruthenium, 2,4,6-trimethylbenzoyl)-phosphine oxide (TPO), Irgacure 2959, Lithium phenyl-2,4,6-trimethylbenzoylphosphinate (LAP), or a combination thereof.

In particular embodiments, the photoinitiator is a photoinitiator that can be excited with visible light. Non-limiting examples of photoinitiators that can be excited with visible light include 2,4,6-trimethylbenzoyl)-phosphine oxide (TPO), Lithium phenyl-2,4,6-trimethylbenzoylphosphinate (LAP), riboflavin, rose bengal or sodium persulphate.

Preferably, the photoinitiator is a photoinitiator that can be excited in the UV spectrum, hence, at a wavelength of from 250 to 450 nm, which may also be referred to as “blue light”. Non-limiting examples of photoinitiators that can be excited with UV irradiation are Irgacure 2959, Lithium phenyl-2,4,6-trimethylbenzoylphosphinate (LAP).

In preferred embodiments, the one or more photoinitiators are water-soluble photoinitiators. Non-limiting examples are ruthenium, Lithium phenyl-2,4,6-trimethylbenzoylphosphinate (LAP), 2,4,6-trimethylbenzoyl)-phosphine oxide (TPO), and other members of the Irgacure photoinitiator family.

In particular embodiments, the crosslinkable composition comprises from 0.1 to 2.0% (w/v), such as from 1.0 to 2.0% (w/v), of one or more photoinitiators.

In particular embodiments, the crosslinkable liquid composition is capable of crosslinking by photocrosslinking such as UV irradiation, by exposure to O₂ or by one or more enzymes. In preferred embodiments, the crosslinkable liquid composition is capable of crosslinking by photocrosslinking or enzymatic crosslinking. Therefore, the crosslinkable liquid composition possesses reactive functionalities that form short oligomer/polymer chains between the macromolecule chains. As described elsewhere in the present specification, the reactive functional groups can be methacrylates, or thiol-ene click chemistry, but are not limited thereto.

In particular embodiments, the crosslinkable liquid composition further comprises one or more therapeutic agents (e.g. an analgesic, an anti-inflammatory agent, an antibiotic, a growth factor to stimulate epithelialization, or a steroid), and/or other agents such as colorants.

Once the crosslinkable liquid composition has been crosslinked, the crosslinked composition preferably does not interfere with the normal functionality of the eye and provides sufficient nutrient and gas exchange to maintain a viable corneal epithelium and stroma. Accordingly, in particular embodiments, the crosslinked composition is permeable to water, nutrients, oxygen, therapeutic agents (e.g. an analgesic, an anti-inflammatory agent, an antibiotic, a growth factor to stimulate epithelialization, or a steroid), and/or growth factors (e.g. exogenous or endogenous growth factors, such as nerve growth factor (NGF))

Preferably, the crosslinked composition is compatible with clinical imaging techniques, such as clinical corneal investigation using a refractometer, optical coherence tomography, Scheimpflug tomography, Placido based tomography device or in vivo confocal imaging.

Accordingly, in particular embodiments, the crosslinkable liquid composition and/or crosslinked composition has a transparency of at least 40%, at least 50%, at least 60%, at least 70%, at least 80% or at least 90%, when measured using light with a wavelength spectrum that is representative of the visual spectrum (400-750 nm), when crosslinked. The transparency may be determined by any means in the art, such as by a microplate reader or spectrophotometer such as described by Rizwan et al. in Sequentially-crosslinked bioactive hydrogels as nano-patterned substrates with customizable stiffness and degradation for corneal tissue engineering applications or by Van Hoorick et al. (Designer Descemet Membranes Containing PDLLA and Functionalized Gelatins as Corneal Endothelial Scaffold, Adv. Healthcare Materials, Vol. 9(6): 2000760; (2020); https://doi.org/10.1002/adhm.202000760)

In particular embodiments, the crosslinkable liquid composition and/or crosslinked composition has a refractive index similar to that of the native corneal stroma. The refractive index may be measured by any means in the art, such as by use of a refractometer.

Upon crosslinking of the crosslinkable liquid composition, the crosslinked composition will hold water within its three-dimensional network of polymers, resulting in the formation of a hydrogel. In particular embodiments, the crosslinked composition has a swelling ratio of 200-1,000%, when fully hydrated. The swelling ratio may be defined as the fractional increase in the weight of the crosslinked composition due to water absorption. The swelling ratio may be influenced by the type of biomaterial, its concentration within the crosslinkable liquid composition and the degree of functionalisation.

One of the main advantages of the method as taught herein over existing techniques is that it provides a long-term solution for treating a refractive error or a chronic or subacute corneal disease involving an irregularity of the cornea. Accordingly, in particular embodiments, the crosslinked composition is stable at body temperature, such at about 37° C., preferably for a period of at least 1 month, at least 2 months, at least 3 months, at least 4 months, at least 5 months, at least 6 months, at least 1 year, at least 5 years, or at least 10 years.

Present inventors have found that by applying the crosslinkable composition to the anterior corneal surface of the eye using a mold, the crosslinkable composition and final crosslinked composition, can be precisely placed onto the anterior corneal surface of the eye while avoiding spilling of the crosslinkable composition over the entire anterior eye surface and/or under the eyelids before crosslinking. Furthermore, the mold may already give a primary shape, diameter and/or preliminary curvature to the final corneal onlay that will be formed by the crosslinked biomaterial. By creating the corneal onlay in situ, it perfectly fits the patient's corneal geometry, compared to a corneal onlay created separately.

In particular embodiments, the mold is an O-ring shaped corneal bath (also referred to as an o-ring). In alternative embodiments, the mold is a contact lens, preferably a contact lens, more preferably a scleral contact lens, that does not adhere to the biomaterial either in uncrosslinked or crosslinked state. In particular embodiments, the mold is a silicone hydrogel contact lens. A contact lens with a converging meniscus-shape allows shaping the crosslinked composition (i.e. biomaterial) into a lens-shape having a lens body (or lens (optical) zone) and a lens edge at the periphery of the lens body (or peripheral zone). Accordingly, the shaped crosslinked composition would use such a contact lens as a mold that is thinner towards its peripheral edges, and hence, typically having a peripherical edge which is less thick than when use is made of a corneal bath. As a result thereof, less biomaterial will need to be removed upon correcting the curvature of the crosslinked composition making the method as taught herein more efficient.

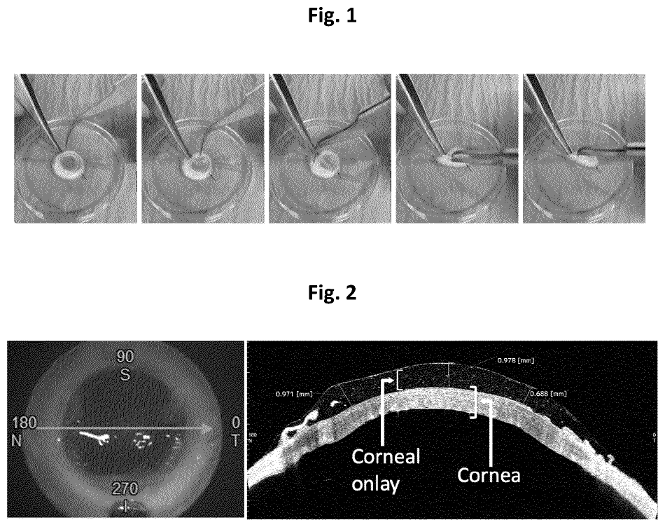

In particular embodiments, the mold is a corneal vacuum suction device. A vacuum suction device acts similarly as an o-ring except that the suction device can be secured on the cornea and consists in different diameters to apply the onlay. In practice, a vacuum suction device (either a dedicated vacuum suction ring or one where the trephine has been taken out of the vacuum suction device) may be vacuum locked on top of the eye, then the crosslinkable composition may be added and irradiated. Then the vacuum suction device is taken off the eye. A vacuum suction device is for instance illustrated in FIG. 1B of Kim et al. (J. Vet Sci, 2015, 16, 349-356). Where the mold is a vacuum suction device, the method may comprise the steps of removing the corneal epithelium, pressing down the vacuum syringe (coupled to the vacuum ring) and placing the ring centrally on the cornea, gently releasing the vacuum syringe to attach the vacuum ring to the eye, placing the crosslinkable fluid composition inside the ring and crosslinking it with the light.

In particular embodiments, the mold is a standard contact lens, which, the inventors have found, when used in the methods of the present invention, inherently generates in an only for which the periphery is thicker than the center.

In particular embodiments, the shape of the mold may be adapted depending on the eye disorder to be treated. For example, for the treatment of farsightedness, biomaterial can be added predominantly centrally of the cornea and for the treatment of nearsightedness, biomaterial can be added predominantly peripherally of the cornea.

The shape of the mold may take into account the swelling of the crosslinkable liquid composition that may occur upon crosslinking and uptake of water by the biomaterial. For example, if a crosslinkable biomaterial is known to swell to twice its size upon crosslinking, and if a thickness of about 50.0 82 m of the crosslinked composition (prior to correcting the curvature) would be desired, the mold may be designed to only allow applying a layer of crosslinkable liquid composition with a thickness of about 25.0 μm. In particular embodiments, the curvature of the contact lens has a back central zone radius of from 8.0 to 15.0 mm, from 8.0 to 14.0 mm, from 8.0 to 13.0 mm, from 8.0 to 12.0 mm, from 8.0 to 11.0 mm, from 8.0 to 10.0 mm.

Preferably, the curvature of the contact lens is so that it allows forming a void space or lens shaped cavity between the anterior surface of the cornea of the subject to be treated and the back surface of the central (optical) zone of the contact lens, while the back surface of the peripheral zone closely aligns with the peripheral zone of the cornea or sclera. This void space or lens shaped cavity can be filled with the crosslinkable composition as described elsewhere herein.

In particular embodiments, the shape of the mold is so that it allows shaping the crosslinked composition as to have an average thickness of from 20.0 μm to 400.0 μm, such as from 50.0 μm to 400.0 μm, from 100.0 μm to 400.0 μm, from 200.0 μm to 400.0 um, or from 100.0 μm to 300.0 μm, prior to correcting the curvature of the crosslinked composition.

In particular embodiments, the mold covers at least 80%, at least 85%, at least 90%, or at least 95%, such as at least 95%; at least 96%, at least 97%, at least 98%, at least 99% or 100%, of the anterior corneal surface of the eye. In particular embodiments, the mold completely covers the anterior corneal surface of the eye.

In particular embodiments, the shape of the mold is so that it allows shaping the crosslinked composition as to have a diameter of from 6.0 to 9.0 mm, such as from 6.0 to 8.0 mm or from 7.0 to 9.0 mm. Present inventors realized that a wider diameter could risk covering the limbal epithelial cells, which differentiate and migrate to become corneal epithelial cells. Therefore, physically covering the limbus forms a risk corneal epithelial cell ingrowth or impedes limbal stem cell differentiation, which is preferably avoided.

In particular embodiments, the mold, such as the contact lens or vacuum suction, has a total diameter (including the diameter of the central zone as well as peripheral zone of the mold) of from 5.0 to 30.0 mm, from 5.0 to 25.0 mm, from 10.0 to 25.0 mm, from 14.0 to 24.0 mm, from 5.0 to 10.0 mm, from 6.0 to 9.0 mm, or from 7.0 to 8.0 mm.

In particular embodiments, if UV irradiation or visible light is used to crosslink the crosslinkable liquid composition, the mold allows the UV or visible light to reach the crosslinkable liquid composition. For example, if the mold is a contact lens, the contact lens allows passage of UV or visible light.

In particular embodiments, the mold is filled with the crosslinkable liquid composition prior to applying the mold and crosslinkable liquid composition to the anterior corneal surface of the eye.

In particular embodiments, the crosslinkable liquid composition is applied onto the anterior corneal surface of the eye in a volume of from 25.0 to 200.0 μl, from 25.0 to 100.0 μl, preferably from 50.0 to 100.0 μl, such as about 50.0 μl. The combination of the mold and the limited amount of volume being used further allows avoiding spilling of the crosslinkable composition over the entire anterior eye surface and/or under the eyelids before crosslinking.

In embodiments, the method may comprise applying said mold onto the anterior corneal surface of the eye either before or after applying said crosslinkable liquid composition into said mold. In embodiments of the uses or methods as taught herein, the mold may be a corneal vacuum suction device, a corneal bath, or a contact lens and the method comprises applying said mold onto the anterior corneal surface of the eye before applying said crosslinkable liquid composition into said mold, cross-linking said composition and removing said mold after cross-linking of said crosslinkable liquid composition.

In particular embodiments, the method comprises maintaining the mold in place on the anterior corneal surface of the eye for the entire period of crosslinking the crosslinkable liquid composition.

In particular embodiments, if the mold is a contact lens, the center of the contact lens is placed onto the center of the anterior surface of the cornea.

In particular embodiments, the method comprises removing the mold after crosslinking the crosslinkable composition (and optionally, where a correction is envisaged, prior to correcting the curvature of the crosslinked composition).

In particular embodiments, the crosslinkable liquid composition is provided as a single layer onto the anterior corneal surface of the eye. In line therewith, in particular embodiments, the crosslinked composition on the anterior corneal surface of the eye consists of a single layer of biomaterial. In particular embodiments, the single layer of biomaterial is homogenous, meaning that it is composed of one and the same biomaterial.

In particular embodiments, prior to applying the mold and the crosslinkable liquid composition onto the anterior corneal surface of the eye, the epithelial cells are removed from the anterior corneal surface to expose the corneal stromal bed for grafting the corneal onlay thereon. The anterior corneal surface may at least in part be debrided of corneal epithelial cells by any means known in the art, such as by use of alcohol delamination, a blunt blade, a diamond burr, a cotton sponge or an Amoils brush. Alternatively the surgeon can opt to remove the epithelium after placing the mold (to only remove the epithelial cells in that area).

In particular embodiments, the method as taught herein does not comprise removal of or damaging the Bowman's layer, the corneal stroma, or a combination thereof prior to applying the crosslinkable liquid composition onto the anterior corneal surface of the eye.

In particular embodiments, the crosslinking is performed by photocrosslinking, by exposure to O₂ or by one or more enzymes such as transglutaminases, transferases, tyrosinases and peroxidases. In case of enzymatic crosslinking, the enzyme and crosslinkable polymer can be mixed upon application in so-called dual barrel syringes which accommodate direct mixing in pre-defined ratios. In preferred embodiments, the crosslinking is performed by photocrosslinking, such as by use of UV light or visible light, more preferably by UV irradiation.

The UV spectrum typically spreads from 250 to 450 nm. Preferably, UV irradiation has a wavelength of from 250 to 450 nm, from 300 to 450 nm or from 300 to 400 nm, such as about 365 nm.

In particular embodiments, the crosslinkable liquid composition is allowed to crosslink until at least 80.0, preferably at least 90.0%, such as 99.9% or 100.0%, of the crosslinkable liquid composition is crosslinked. In particular embodiments, the crosslinkable liquid composition is being crosslinked using UV irradiation for a period of at least 10 minutes, at least 20 minutes, at least 30 minutes, or at least 40 minutes, preferably at least 30 minutes.

In particular embodiments, the total dose of UV used during UV irradiation is at least 0.7, at least 0.8, at least 0.9, at least 1.0, at least 2.0, at least 3.0, at least 4.0 or at least 5.0 Joule (J)/cm², preferably at least 1.0 Joule/cm² or at least 5 Joule/cm², such as about 5.4 J/cm². The power or intensity of irradiation ranges from 1 to 10 mW/cm², which is inversely correlated to the irradiation time.

In the method of present invention, the crosslinking is performed in situ, onto the anterior corneal surface of the eye. Accordingly, in particular embodiments, the crosslinking is performed at body temperature, such as at a temperature of from 35.0° C. to 40.0° C. or from 36.0° C. to 38.0° C.

The method as taught herein provides a user-friendly and long-term treatment of the refractive error or the chronic or subacute corneal disease involving an irregularity of the cornea. As the crosslinked composition on the anterior corneal surface of the eye is resistant to biodegradation, is less likely to result in post-procedure complications such as dry eye disease, pain or regression of the applied correction, and does not need to be replaced regularly.

In particular embodiments, the crosslinked composition is resistant to biodegradation for a period of at least 6 months, preferably at least 12 months. Resistance to biodegradation may be controlled by the type of monomer/oligomer of the biomaterial, the concentration of the polymer in the crosslinkable liquid composition, the molecular weight of the monomer of the biomaterial and/or the degree of substitution of the monomer/oligomer of the biomaterial. The degree of crosslinking can also be controlled by the crosslinking parameters, such as the UV crosslinking parameters like the duration and light intensity. The resistance to biodegradation can also be tuned by mixing different types of polymers. For example, polyethylene glycol (PEG)-based hydrogels are typically more resistant to biodegradation than protein-based hydrogels, which can be mixed in.

In particular embodiments, the crosslinkable liquid composition is resistant to degradation by matrix metalloproteinases (MMPs) of the corneal epithelium, such as MMP-1, MMP-2, MMP-3, MMP-9, or a combination thereof.

Crosslinking of the crosslinkable liquid composition onto the anterior corneal surface of the eye leads to adherence between the crosslinked composition or corneal onlay and the anterior corneal surface of the eye through the formation of covalent bonds with the amino acids in the corneal collagen. In particular embodiments, the posterior surface of the crosslinked composition adheres to the Bowman's membrane of the eye with an adhesion strength of at least 10.0 kPa, and preferably with an adhesion strength from 10.0 to 100.0 kPa. The adhesion strength may be determined by any methods known in the art, such as by a lap shear test with a universal testing machine according to the ASTM F2255 using gelatin coated glass slides.

In particular embodiments, the posterior surface of the crosslinked composition contacts the Bowman's membrane of the eye.

In particular embodiments, the crosslinked composition is flexible. The flexibility of the crosslinked composition may be altered by changing the type of biomaterial used, its concentration in the crosslinkable liquid composition, its degree of substitution or its molecular weight. Preferably, the flexibility of the crosslinked composition is similar to native cornea.

In particular embodiments, it can be of interest to correct the curvature of the onlay once the crosslinkable composition has been crosslinked onto the anterior corneal surface of the eye. More particularly, the crosslinked composition may be reshaped for optimal and/or patient-specific treatment of the eye disorder, such as a patient-tailored vision correction. For example, the radius of curvature of the crosslinked composition may be corrected, such as by use of photoablation, to treat the eye disorder. Reshaping of the crosslinked composition may change the refractive properties of the so-treated eye in a desired manner to correct the eye disorder, such as the refractive error.

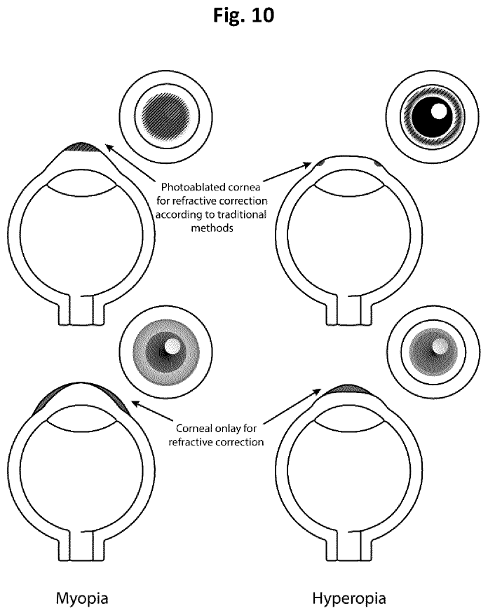

The person skilled in the art will understand that depending on the eye disorder to be treated, the radius of curvature of the crosslinked composition may be corrected differently. For example, for the treatment of farsightedness, biomaterial can be maintained predominantly centrally and for the treatment of nearsightedness, biomaterial can be maintained predominantly peripherally. The ablation depth of the corneal onlay is correlated to the envisaged refractive correction and calculated similarly to current refractive laser surgery. Personalized treatment profiles, also known as nomograms, are generated and transferred to a laser for treatment. It is noted that the mode of refractive correction is different from current strategies because of the inherent nature of the corneal onlay technology. Whereas in case of myopia, laser refractive surgery subtracts peripheral tissue, a corneal onlay corrects myopia by adding peripheral tissue. Vice versa, laser refractive surgery corrects hyperopia by tissue dissection mid-peripherally, while a corneal onlay corrects this by adding tissue centrally (FIG. 10).

In particular embodiments, the crosslinked composition is photoablated to obtain a refractive correction, preferably a spherical refractive correction, in the range of from −20 diopters to +10 diopters. In particular embodiments, the crosslinked composition is photoablated such that the crosslinked composition comprises at least a central portion having a substantially uniform thickness extending from the lower surface to the upper surface of the crosslinked composition such that the crosslinked composition has an optical power within a range from −20 diopters to about +10 diopters, preferably from −10 diopters to about +5 diopters, along at least the inner portion of the crosslinked composition. In particular embodiments, the cornea of the eye is not photoablated.

In particular embodiments, the crosslinked composition is photoablated to obtain a non-spherical shape when the eye disorder is astigmatism.

In particular embodiments, the crosslinked composition is photoablated to obtain a lens-shape, such as having a thickness of from 10.0 to 50.0 μm at the periphery of the outer portion of the crosslinked composition and extending to the central portion of the crosslinked composition with an increasing thickness to from 30.0 to 100.0 μm.

In particular embodiments, the upper surface of the crosslinked biomaterial is being photoablated to shape the upper surface.

Furthermore, the person skilled in the art will also understand that the possible deswelling of the crosslinked composition upon overgrowth of the epithelial cells should be taken into account when determining the amount of crosslinked composition that will be removed from the eye to treat the eye disorder.

Photoablation may be performed using a laser, such as an excimer laser.

In particular embodiments, the correcting of the curvature of the crosslinked composition does not comprise removing corneal tissue, such as corneal stromal tissue, such as by photoablation.

In particular embodiments, no correction of the curvature of the crosslinked material is necessary. This can be the case where the layer of material is very thin and/or where a mold can be used which ensures exactly the desired shape and thickness of the material after crosslinking. In particular embodiments photoablation of the corneal onlay is not performed when the principal aim is to treat chronic or subacute corneal disease.

In particular embodiments, the method as taught herein is reversible, meaning that the crosslinked composition may be completely removed from the anterior corneal surface of the eye, if needed, such as by photoablation, hydrodissection, microkeratome or manual dissection.

After correcting the curvature of the crosslinked composition, the corneal epithelium may spontaneously reform originating from the corneal limbus, the corneal scleral transition zone. Overgrow of the crosslinked composition by the corneal epithelium typically occurs within 1 to 2 weeks after correcting the curvature of the crosslinked composition. In particular embodiments, one or more therapeutic agents, such as NGF, are administered to the eye to improve regrowth of the corneal epithelium.

The subject may post-operatively be treated with therapeutic agents that reduce pain and/or inflammation, such as corticosteroids, and/or antibiotics.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and scope of the appended claims.

The herein disclosed aspects and embodiments of the invention are further supported by the following non-limiting examples.

### EXAMPLES

### Example 1. Crosslinking Biomaterial to the Anterior Surface of a Cornea of a Human Donor Eye

### 1.1. Material and Methods

Research grade cadaveric human donor eyes were disinfected in povidone iodine solution for one minute. The donor eyes were subsequently washed three times 5 minutes in 1× phosphate buffered saline (PBS). Next, corneas were dissected from the doner eyes using a circular trephine to obtain a corneascleral ring and kept in PBS until further use. A photocrosslinkable solution (also referred to as polymer solution) was prepared from gelatin methacryloyl (GelMA; Advanced Biomatrix #5208). The polymer solution (10% (w/v)) was warmed at 37° C. and appropriate concentration of photoinitiator was mixed in (GelMA 10% (w/v) with 2 mol% irgacure 2595 (Merck 410896)). 50 μL of polymer solution+photoinitiator was loaded in a silicone hydrogel contact lens and placed on top of the human donor cornea. The cornea with polymer was placed in the AnalytikJena crosslinker (365 nm) with an accumulated irradiation dosage of 5400 mJ/cm² for about twenty minutes. The contact lens was removed and the eye is hydrated. Using forceps, the adhesion of the polymerized material to the cornea was investigated. The cornea with onlay was imaged using Optical Coherence Tomography (CASIA2 OCT apparatus) while holding with a forceps. The corneal onlay was measured using the internal CASIA2 software.

### 1.2. Results

It was judged by a corneal surgeon with expertise in corneal sealants and tissue glues that the methacrylated gelatin was firmly attached to the cornea surface (FIG. 1). Afterwards, imaging with Optical Coherence Tomography showed proper adhesion of the onlay to the cornea and a central thickness of around 1000 μm and a thinning peripheral region (FIG. 2). Based on keratometry, the increase in corneal thickness resulted in a changed refractive power of the cornea (data not shown).

### Example 2. Gel Fraction, Water Uptake and Transparency of Methacrylated Gelatin

2.1 Material and methods

Gelatin methacrylate (GelMa) was used at different concentrations (5, 10 and 15 w/v %) using phosphate buffered saline (1×) as a solvent.

Different photoinitiators in different doses were added to the GelMa solution and stored in amber vials to protect from the light:
Irgarcure 2595 (12595): 0.5% (“Irga1”) and 1% (“Iirga2”)
Lithium phenyl-2,4,6 trimethylbenzoylphosphinate (LAP): 0.125% (“LAP1”) and 0.250% (“LAP2”)

A drop of the GelMa solution+photoinitiator was placed on a glass petridish.

The GelMa solution+photoinitiator was crosslinked at 365 nm with a total dose of 5.4 J/cm².

The gel fraction is defined as the weight ratio of dried network polymer (crosslinked material) to that of the polymer before washing by solvent. The weight was determined using a Sartorius BP211D microbalance.

The swelling ratio is defined as the fractional increase in the weight of the hydrogel due to water absorption. The weight increase was determined using a Sartorius BP211D microbalance at determined intervals.

Transparency (% of light transmittance) is converted from absorbance values with the following equation: % T=10^{(2-A)} (% T=transparency; A=absorbance). Absorbance values were measured with a Tecan multimode microplate reader.

### 2.2 Results

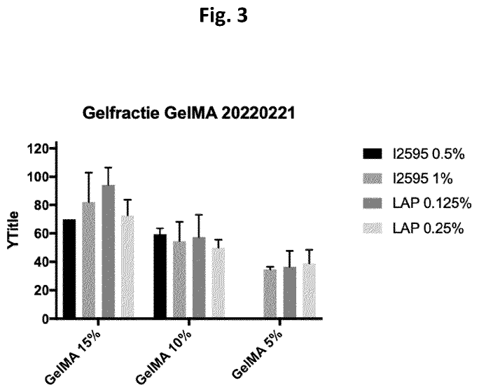

FIG. 3 shows that the gel fraction of GelMA increased with polymer concentration due to the increased polymer density for crosslinking.

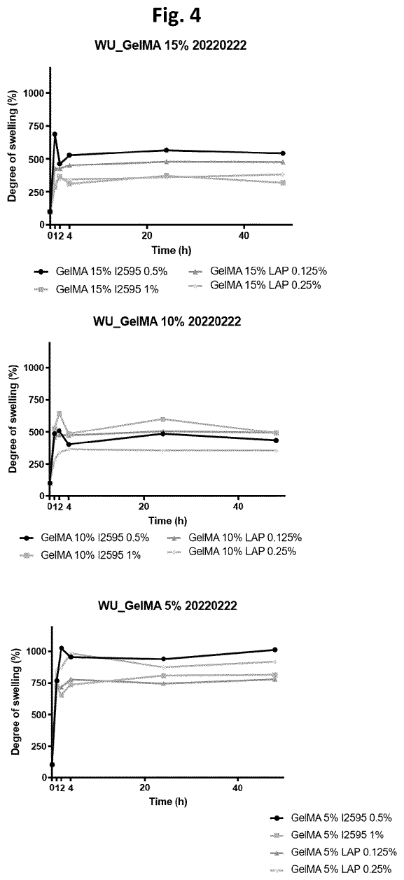

FIG. 4 shows that the water uptake for GelMA in different concentrations with different photoinitiators. Hydrogels with the lowest concentration of GelMA exhibited the highest water uptake and vice versa, which can be explained by the increased gel fraction and thus viscosity of the hydrogel with increasing polymer concentration. All concentrations reached an equilibrium after 4 hours.

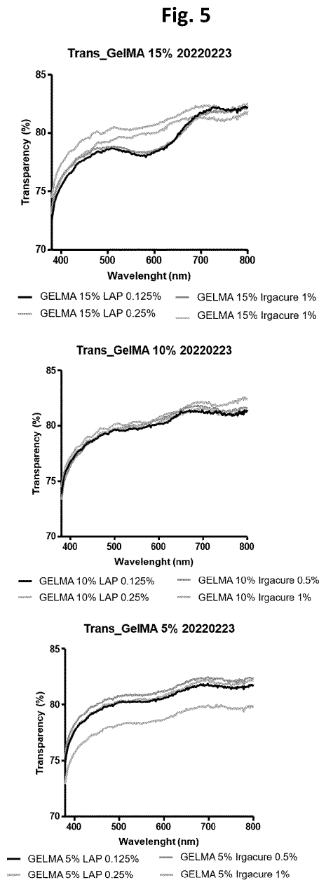

FIG. 5 shows that the transparency of GelMA is at least 75% when measured over the visual spectrum. No significant transparency difference was observed for any polymer or photoinitiator concentration.

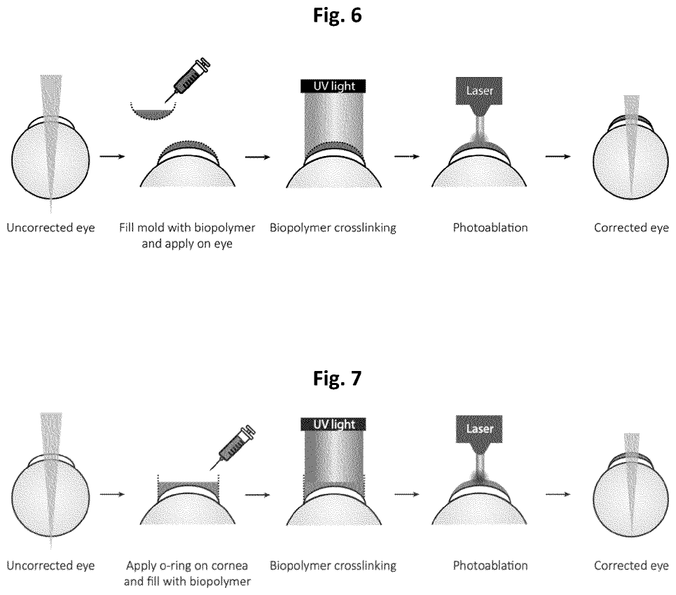

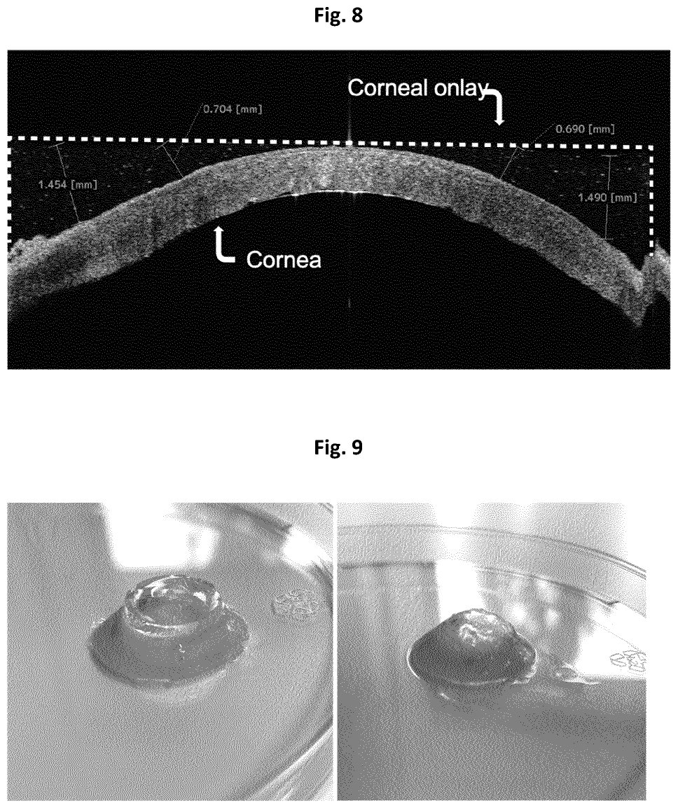

### Example 3. Exemplary Method of Treating a Refractive Error of the Eye, Such as Farsightedness, as Illustrated in FIG. 6

1. The refractive error of the patient is diagnosed according to established methods.
2. The patient's corneal epithelium is removed using diluted alcohol, a blunt blade or an Amoils brush.
3. The uncrosslinked polymer (dissolved photocrosslinkable polymer with final concentration of photoinitiator) is applied on top of the eye with a mold, such as a contact lens or vacuum suction device for the entire crosslinking period. The volume of the solution is preferably equal to or less than 50 μL.
4. The treated eye is irradiated to fully crosslink and adhere the corneal onlay. The accumulated dosage of UV light corresponds or is less than the current UV irradiation protocols for keratoconus treatments (e.g. 5.4 J/cm²). The irradiation time is dependent of the chosen intensity
   a. E.g. if the photoinitiator used is Irgacure 2595: ultraviolet light of 365 nm
   b. E.g. if the photoinitiator used is LAP: 400-450 nm visible light is also possible
5. The polymer covalently adheres to extracellular matrix of the eye during the crosslinking process and is allowed to rehydrate (isotonic eye drops can be administered). The equilibration period may be at least 4 hours. The mold, such as the contact lens or vacuum suction device, is removed (either before or after the equilibrium period) before laser refractive surgery.
6. The patient is subjected to refractive laser surgery where the excimer laser only cuts away the corneal onlay and not the cornea tissue.
7. The patient is preferably treated with corticosteroids and antibiotics similarly to what is administered for refractive surgery
8. The corneal epithelium will spontaneously overgrow the corneal onlay within 1-2 weeks post operatively.

### Example 4. Exemplary Method of Treating a Refractive Error of the Eye, Such as Farsightedness, as Illustrated in FIG. 7

1. The refractive error of the patient is diagnosed according to established methods.
2. The patient's corneal epithelium is removed using diluted alcohol, a blunt blade or an Amoils brush.
3. An o-ring or vacuum suction device is placed on top of the cornea of the patient
4. The uncrosslinked polymer (dissolved photocrosslinkable polymer with final concentration of photoinitiator) is applied in the o-ring or vacuum suction device. The volume of the solution is preferably equal to or less than 50 μL.
5. The treated eye is irradiated to fully crosslink and adhere the corneal onlay. The accumulated dosage of UV light corresponds or is less than the current UV irradiation protocols for keratoconus treatments (e.g. 5.4 J/cm²). The irradiation time is dependent of the chosen intensity
   a. E.g. if the photoinitiator used is Irgacure 2595: ultraviolet light of 365 nm
   b. E.g. if the photoinitiator used is LAP: 400-450 nm visible light is also possible
6. The polymer covalently adheres to extracellular matrix of the eye during the crosslinking process and is allowed to rehydrate (isotonic eye drops can be administered). The equilibration period may be at least 4 hours. The o-ring or vacuum suction device is removed (typically before or during the equilibration period).
7. The patient is subjected to refractive laser surgery where the excimer laser only cuts away the corneal onlay and not the cornea tissue.
8. The patient is preferably treated with corticosteroids and antibiotics similarly to those administered after refractive surgery
9. The corneal epithelium will spontaneously overgrow the corneal onlay within 1-2 weeks post operatively.

1. A crosslinkable liquid composition for use in the treatment of an eye disorder in an eye of a subject, wherein the treatment comprises a method comprising the steps of
applying said crosslinkable liquid composition onto an anterior corneal surface of said eye comprising said eye disorder;
crosslinking the crosslinkable liquid composition on the anterior corneal surface of the eye, thereby obtaining a crosslinked composition on the anterior corneal surface of the eye; and optionally,
correcting the curvature of the crosslinked composition,
wherein the eye disorder is a refractive error or a chronic or subacute corneal disease involving an irregularity of the cornea, and
wherein the method comprises introducing said crosslinkable liquid composition into a mold which is positioned onto the anterior corneal surface of the eye.

2. The crosslinkable liquid composition according to claim 1, wherein the mold is a corneal vacuum suction device, a corneal bath or a contact lens and the method comprises either applying said mold onto the anterior corneal surface of the eye either before or after applying said crosslinkable liquid composition into said mold.

3. The crosslinkable liquid composition according to claim 1 or 2, wherein the method comprises removing the mold after crosslinking the crosslinkable composition.

4. The crosslinkable liquid composition according to any one of claims 1 to 3, wherein wherein the anterior corneal surface is debrided of corneal epithelial cells before or after placing the mold on the cornea.

5. The crosslinkable liquid composition according to any one of claims 1 to 4, wherein the crosslinkable liquid composition is applied as a single layer.

6. The crosslinkable liquid composition according to any one of claims 1 to 5, wherein the crosslinkable liquid composition comprises a crosslinkable biomaterial and optionally one or more photoinitiators.

7. The crosslinkable liquid composition according to claim 6, wherein the crosslinkable biomaterial is selected from the group consisting of protein-based polymers, polysaccharide-based polymers and synthetic polymers and combinations thereof.

8. The crosslinkable liquid composition according to claim 7, wherein the protein-based polymer is selected from collagen, gelatin, fibrinogen, silk fibroin, or a mussel inspired 3,4-dihydroxyphenyl-L-alanine (DOPA) polymer.

9. The crosslinkable liquid composition according to claim 8, wherein the protein-based polymer gelatin methacryloyl (GelMa).

10. The crosslinkable liquid composition according to claim 7, wherein the synthetic polymer is selected from include polyethylene glycol (PEG), poly(e-caprolactone) (PCL) and poly(vinylalcohol).

11. The crosslinkable liquid composition according to any one of claims 1 to 10, wherein the crosslinking is performed by photocrosslinking, by exposure to O₂ or by one or more enzymes, preferably by photocrosslinking such as by UV irradiation.

12. The crosslinkable liquid composition according to any one of claims 1 to 11, wherein the crosslinked composition has a diameter of from 6.0 to 9.0 mm and a thickness of from 20.0 μm to 400.0 μm prior to correcting the curvature of the crosslinked composition.

13. The crosslinkable liquid composition according to any one of claims 1 to 12, wherein the crosslinked composition is resistant to biodegradation for a period of at least 6 months, preferably at least 12 months.

14. The crosslinkable liquid composition according to any one of claims 1 to 13, wherein the refractive error is selected from the group consisting of myopia, hyperopia, astigmatism and presbyopia.

15. The crosslinkable liquid composition according to any one of claims 1 to 14, wherein the chronic or subacute corneal disease involving an irregularity of the cornea is selected from the group consisting of corneal ulcer, corneal erosion, a corneal ectatic disorder or a corneal irregularity caused by trauma or epithelial basement membrane dystrophy.

16. A method of treating an eye disorder in an eye of a subject, comprising
applying a crosslinkable liquid composition onto an anterior corneal surface of said eye comprising said eye disorder, optionally wherein the anterior corneal surface has been debrided of corneal epithelial cells;
crosslinking the crosslinkable liquid composition on the anterior corneal surface of the eye, thereby obtaining a crosslinked composition on the anterior corneal surface of the eye; and optionally,
correcting the curvature of the crosslinked composition,
wherein the eye disorder is a refractive error or a chronic or subacute corneal disease involving an irregularity of the cornea, and
wherein the method comprises introducing said crosslinkable liquid composition into a mold which is positioned onto the anterior corneal surface of the eye.

17. The method according to claim 16, wherein the mold is a corneal bath or a contact lens and the method comprises either applying said mold onto the anterior corneal surface of the eye either before or after applying said crosslinkable liquid composition into said mold.

18. The method according to claim 16 or 17, wherein the method comprises removing the mold after crosslinking the crosslinkable composition.

19. The method according to any one of claims 16-18, wherein the crosslinkable liquid composition is applied as a single layer.

20. The method according to any one of claims 16-19, wherein the crosslinkable liquid composition comprises a crosslinkable biomaterial and optionally one or more photoinitiators.

21. The method according to claim 20, wherein the crosslinkable biomaterial is selected from the group consisting of protein-based polymers, polysaccharide-based polymers and synthetic polymers.

22. The method according to any one of claims 16 to 21, wherein the crosslinking is performed by photocrosslinking, by exposure to O₂ or by one or more enzymes, preferably by photocrosslinking such as by UV irradiation.

23. The method according to any one of claims 16 to 22, wherein the crosslinked composition has a diameter of from 6.0 to 9.0 mm and a thickness of from 20.0 μm to 400.0 μm prior to correcting the curvature of the crosslinked composition.

24. The method according to any one of claims 16 to 23, wherein the crosslinked composition is resistant to biodegradation for a period of at least 6 months, preferably at least 12 months.

25. The method according to any one of claims 16 to 24, wherein the refractive error is selected from the group consisting of myopia, hyperopia, astigmatism and presbyopia.

26. The method according to any one of claims 16 to 25, wherein the chronic or subacute corneal disease involving an irregularity of the cornea is selected from the group consisting of corneal ulcer, corneal erosion, a corneal ectatic disorder or a corneal irregularity caused by trauma or epithelial basement membrane dystrophy.
